(19) United States
(12) Plant Patent Application Publication
(10) Pub. No.: US 2001/0025381 P1
(43) Pub. Date: Sep. 27, 2001

(54) Miniature rose variety 'POULra007'

(75) Inventors: L. Pernille Olesen, Fredensborg (DK); Mogens N. Olesen, Fredensborg (DK)

Correspondence Address: Mogens N. Olesen, Poulsen Roser Pacific, Inc., 2550 Old Military Road, Central Point, OR, 97502, US

(73) Assignee: Poulsen Roser ApS

(21) Appl. No.: 09/780,135

(22) Filed: Feb. 8, 2001

(30) Foreign Application Priority Data
Feb. 11, 2000 (EP) 2000/0236
Jun. 6, 2000 (CA) 00-2239

(51) Int. Cl. A01H 5/00

(52) U.S. Cl. PLT/122

(57) ABSTRACT

A new miniature rose plant which has abundant, red flowers and attractive foliage. The variety successfully propagates from softwood cuttings and is suitable for year round production in commercial glasshouses. This new and distinct variety has shown to be uniform and stable in the resulting generations from asexual propagation.

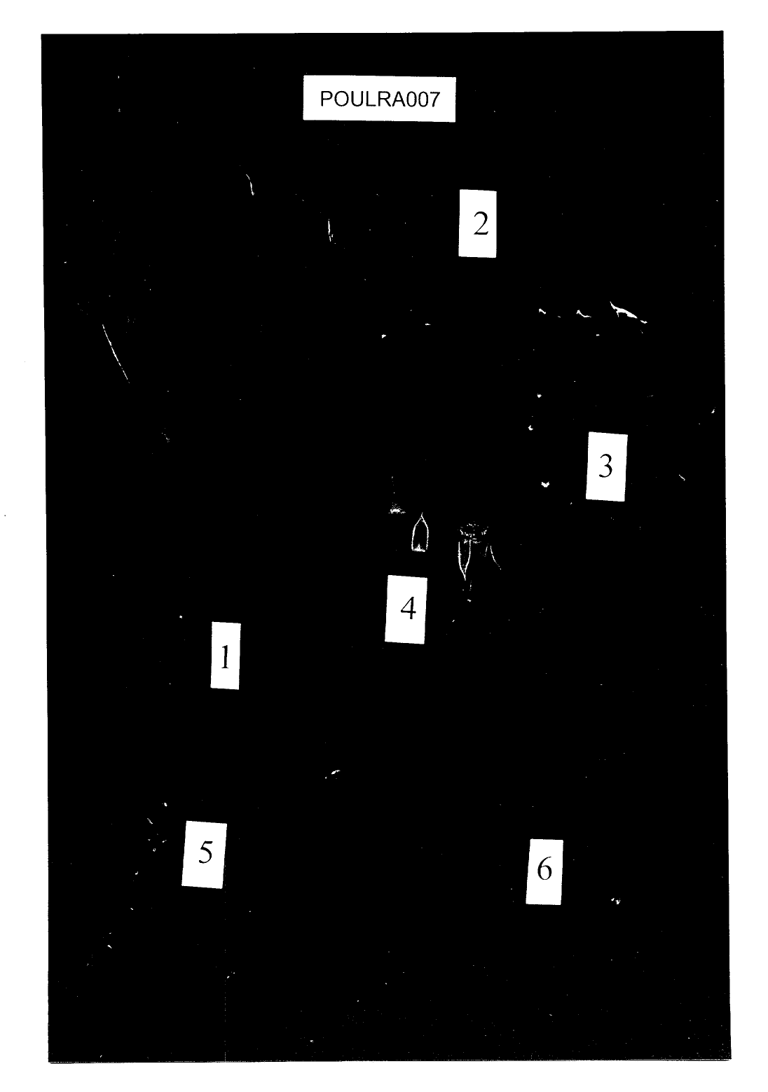

### SUMMARY OF THE INVENTION

The present invention constitutes a new and distinct variety of miniature rose plant which originated from a controlled crossing between ‘POULrac’, described and illustrated in U.S. Plant Pat. No. 11,543 issued Oct. 3, 2000 and ‘POULisab’, described and illustrated in U.S. Plant patent application Ser. No. 09/270,177 dated Mar. 15, 1999. The two parents were crossed in July 1997 and the resulting seeds were planted in a controlled environment. The new variety is named ‘POULra007’.

The new rose may be distinguished from its seed parent, ‘POULrac’, by the following combination of characteristics:

1. The growth habit of ‘POULrac’ is smaller.

2. The size of the open bloom of ‘POULrac’ is smaller than ‘POULra007’.

3. The seed parent has more flowers per stem.

The new variety may be distinguished from its pollen parent, ‘POULisab’ created by the same inventors, by the following combination of characteristics:

1. ‘POULisab’ is a hybrid tea and much larger in habit.

2. The foliage of the pollen parent is much larger when compared to ‘POULra007’.

The objective of the hybridization of this rose variety for commercial culture was to create a new and distinct variety with unique qualities, such as:

1. Uniform and abundant red flowers;

2. Vigorous and compact growth;

3. Year-round flowering under glasshouse conditions;

4. Suitability for production from softwood cuttings in pots;

5. Durable flowers and foliage which make a variety suitable for distribution in the floral industry.

This combination of qualities is not present in previously available commercial cultivars of this type and distinguish ‘POULra007’ from all other varieties of which we are aware.

As part of their rose development program, L. Pernille Olesen and Mogens N. Olesen germinated the seeds from the aforementioned hybridization in winter 1997 and conducted evaluations on the resulting seedlings in a controlled environment in Fredensborg, Denmark.

‘POULra007’ was selected by the inventors as a single plant from the progeny of the hybridization in spring 1998.

Asexual reproduction of ‘POULra007’ by cuttings and traditional budding was first done by L. Pernille and Mogens N. Olesen in their nursery in Fredensborg, Denmark in June 1998. This initial and other subsequent propagations conducted in controlled environments have demonstrated that the characteristics of ‘POULra007’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, and stems of ‘POULra007’. Specifically illustrated in SHEET 1:

1. Stem showing branching and the attachment of leaves, buds, and peduncles;

2. Flower bud, partially opened bud, and open bloom;

3. Flower petals, detached;

4. Sepals, receptacle, and pedicel;

5. Flowering stem as well as a bare stem exhibiting thorns;

6. Leaves.

### DETAILED DESCRIPTION OF THE VARIETY

The following is a description of ‘POULra007’, as observed in its growth in Burlington, Ontario, Canada and Fredensborg, Denmark. Color references are made using The Royal Horticultural Society (London, England) Colour Chart, 1995, except where common terms of color are used.

For a comparison, several physical characteristics of the rose variety ‘POULrac’, a rose variety from the same inventors described and illustrated in U.S. Plant Pat. No. 11,543 and issued on Oct. 3, 2000 are compared to ‘POULra007’ in Chart 1.

CHART 1

| | ‘POULra007’ | ‘POULrac’ |
|---|---|---|
| Bud color at | Red-Purple Group | Red Group 53A to |
| ¼ open | 59A | Red-Purple Group |
| | | 60B |
| Upper petal | Red Group 45B | Between Red Group |
| surface, open | | 46A and Red- |
| bloom | | Purple Group 60B |
| Size of pot | 20-22 cm (h) × | 16-18 (h) × |
| plant | 18-20 cm (w) | 16-18 (w) |

1. A new and distinct variety of rose plant of the miniature class, substantially as herein illustrated and described as a distinct and novel rose variety due to its abundant, red flowers, vigorous growth, compact habit, suitability for production from softwood cuttings in pots, and durable flowers and foliage which make the variety suitable for distribution in the floral industry.
